(19) United States
(12) Patent Application Publication
(10) Pub. No.: US 2011/0262551 A1
(43) Pub. Date: Oct. 27, 2011

(54) COSMETIC COMPOSITION

(75) Inventors: Carine NIZARD, Ivry Sur Seine (FR); Jean-Hubert CAUCHARD, Orleans (FR); Kristell LAZOU, Orleans (FR); Jean-Christophe ARCHAMBAULT, Meung Sur Loire (FR); Emmanuelle NOBLESSE, Donnery (FR); Frédéric BONTE, Orleans (FR); Sylvianne SCHNEBERT, Olivet (FR); Eric PERRIER, Les Cotes D'Arey (FR)

(73) Assignee: LVMH RECHERCHE, Saint Jean de Braye (FR)

(21) Appl. No.: 13/019,145

(22) Filed: Feb. 1, 2011

(30) Foreign Application Priority Data
Feb. 3, 2010 (FR) 1050758

(51) Int. Cl. A61K 8/96; A61Q 19/08

(52) U.S. Cl. 424/537

(57) ABSTRACT

A cosmetic composition includes, as a cosmetic active agent, a honey from clover (Trifolium repens) or an extract of this honey. A method uses a cosmetic composition, of a honey from clover (Trifolium repens), or an extract of this honey, as an active agent for preventing or delaying the appearance of the signs of aging of the skin or slowing the effects thereof, in particular for restructuring the epidermis, toning the skin and/or promoting the attenuation or resorption of wrinkles or lines, notably on the face and neck. A method of cosmetic care also uses this cosmetic composition.

This application claims benefit of Serial No. 1050758, filed 3 Feb. 2010 in France and which application is incorporated herein by reference. A claim of priority, to the extent appropriate is made.

The invention relates to the use, in a cosmetic composition, of a honey from clover (Trifolium repens) or of an extract of this honey, as active agent for preventing or delaying the appearance of the signs of aging of the skin or for attenuating their effects, as well as a cosmetic composition containing an active agent and a method of cosmetic care comprising application thereof.

### PRIOR ART

As is well known, skin aging results from genetic factors but also from environmental factors, for example exposure to sunlight, and is manifested by simultaneous molecular, cellular, histological and clinical changes in the epidermis and the dermis.

It is found that during aging, changes occur in the mechanisms maintaining the life of the cell, until apoptosis or “genetically programmed cell death”. These disturbances cause a decrease in cellular proliferation as well as a progressive decline of cellular activity, reflected in changes of the cell cycle such as oxidation, deterioration of intercellular communications, or the formation of free radicals.

Thus, notably we observe a decrease in thickness of the epidermis and a decrease in the size of the epidermal crests, which is reflected in flattening of the dermal-epidermal junction, leading in turn to reduced cohesion at the interface of the epidermis and the dermis.

This cutaneous atrophy is also gradually reflected in changes of the extracellular matrix connected with a decrease in production of collagen and of the elastin fibres of the skin, as well as a decline in the levels of proteoglycans, glycosaminoglycans and fibronectin.

All these phenomena lead to increased fragility of the skin, associated with a deterioration of its protective and barrier functions, in particular with respect to the environmental stresses to which it is subjected, and of its mechanical properties (loss of elasticity, making laughter lines more visible).

Repeated mechanical stresses also lead to changes in gene expression, leading for example to overactivation of the metalloproteinases, which accentuate skin aging by a local decrease in substance, resulting in deepening of wrinkles and loss of firmness.

Conversely, healing is itself a process of tissue repair notably of the dermis and epidermis, resulting in a re-epithelialization of the wound.

This process comprises several stages:
a phase of tissue repair, corresponding to the migration and proliferation of fibroblasts, which is dependent on cytokines, including TGF-beta (transforming growth factor-beta), and to the synthesis of a new extracellular matrix composed of collagen, fibronectins and proteoglycans.
a phase of epithelialization of the wound comprising the migration of epithelial cells (keratinocytes), then their differentiation to reform the epidermis.

The activity of honey in the care of burns, of superficial wounds or of ulcers is known.

Honey permits quick and clean healing of wounds and rapid replacement of damaged tissues.

It stimulates the formation of granulation tissues, the development of neovascularization and fibroblast proliferation, and thus speeds up the epithelialization of wounds (Molan, Primary Intentions, 1998, December, 148-158; Efem, Br. J. Surg., 1988, 75, 679-681).

Notably, the use of thyme honey in the care of surgical wounds is known (Descottes, Phytothérapie, 2009, 7, 112-116).

### AIMS OF THE INVENTION

As already noted, as the mechanisms of cellular proliferation and of synthesis of the structural components of the skin gradually decline during aging, their stimulation is therefore a strategy that aims to reproduce the process of healing of damaged skin, to obtain the same effects.

A main aim of the invention is to identify active agents that stimulate targets involved in the healing processes, to obtain an increase in cell density in the dermis and epidermis, and remodelling of the extracellular matrix, thus contributing to improvement of the mechanical and functional properties of the skin.

A main aim of the invention is to supply a novel cosmetic active agent having good healing power, of natural origin, and which is environment-friendly.

Another main aim of the invention is to find a novel cosmetic active agent that is inexpensive, and is available in sufficient quantity for the cosmetic industry.

### SUMMARY OF THE INVENTION

The applicant has discovered that honey from clover (Trifolium repens) significantly stimulates the expression of genes coding for the synthesis of cytokines involved in the healing processes, and that in addition it makes it possible to accelerate the proliferation of fibroblasts in artificially damaged dermis.

Based on these results, it is possible to envisage the use of this particular active agent as a cosmetic active agent for stimulating the processes of repair of the superficial tissues of healthy, undamaged skin, in particular of the dermis and epidermis, so as to restore its mechanical and functional properties.

Improvement of the mechanical properties of the skin is notably reflected in a visible attenuation of the wrinkles and lines of the skin, in particular of the face and neck.

### DETAILED DESCRIPTION OF THE INVENTION

The applicant discovered, surprisingly, that clover honey acts more effectively than thyme honey or other active agents known to have healing power, with respect to the expression of genes involved in the synthesis of cellular factors produced during the healing processes. It is also more effective in stimulating the proliferation of fibroblasts in the dermis of artificially damaged skin.

Clover honey is thus an active agent that is particularly effective for restoring cellular activity, the progressive decline of which induces loss of elasticity of the skin, leading to the formation of wrinkles or lines.

Clover honey is widely available commercially.

Clover honey comprises predominantly, locked in its mass, pollen grains whose shape and size are characteristic of the plant species Trifolium repens.

Thus, a first object of the present application relates to the use, in a cosmetic composition, of a honey from clover (Trifolium repens), or of an extract of this honey, as an active agent for preventing or delaying the appearance of the signs of aging of the skin or to slow down their effects, in particular for restructuring the epidermis, for toning the skin and/or for promoting the attenuation or resorption of wrinkles or lines, notably on the face and neck.

Preferably, clover honey, or an extract of this honey is used as a cosmetic anti-wrinkle agent.

A particularly preferred clover honey is that produced in New Zealand.

A second object of the invention relates to a cosmetic composition comprising, as cosmetic active agent, a honey from clover (Trifolium repens) or an extract of this honey.

More particularly, the invention relates to an anti-aging or anti-wrinkle cosmetic composition comprising a honey from clover (Trifolium repens) or an extract of this honey, as anti-wrinkle or anti-aging active agent.

The composition can also comprise one or more unifloral honeys from other plants, preferably a unifloral honey from thyme (Thymus vulgaris) or a honey from manuka (Leptospermum scoparium), and/or one or more polyfloral honeys, and/or one or more extracts of these honeys and/or one or more products from apiculture, such as royal jelly.

The preparation of a honey extract is well known by a person skilled in the art.

A preferred extract is obtained using polar solvents.

As the polar solvent or mixture of polar solvents, advantageously a solvent or a mixture of solvents is selected from water, a C₁-C₄ alcohol, for example ethanol, a C₂-C₆ glycol preferably selected from glycerol, butylene glycol and propylene glycol and mixtures thereof.

According to a preferred embodiment of the invention, extraction is carried out using an aqueous-alcoholic or aqueous-glycolic mixture.

The properties of clover honey can be obtained or improved in cosmetic compositions, in which it is combined with other active agents in the form of purified molecules and/or of extracts from plants whose cosmetic effects are similar and/or complementary to those of said honey.

In addition to the aforementioned clover honey, the cosmetic composition can thus comprise one or more other cosmetic active agents selected from substances having anti-aging activity; substances having a depigmenting activity or a skin lightening activity; substances having a slimming activity; substances having a hydrating activity; substances having a calming, soothing or relaxing activity; substances stimulating the cutaneous microcirculation for improving radiance, in particular of the face; substances having sebo-regulatory activity for the care of greasy skin; substances intended to clean or purify the skin; substances having anti-radical activity.

Thus, a cosmetic composition of the invention advantageously comprises one or more other substances selected from:
molecules promoting cellular renewal such as vitamin A, retinol and/or its esters; alpha or beta-hydroxy acids such as acids from fruits, notably malic, glycolic or citric acid, salicylic acid or its esters, gentisic acid or its esters, in particular tocopherol gentisate.
molecules or extracts stimulating the firmness of the skin such as peptides stimulating the synthesis of collagen, in particular of collagen of type I, II, IV or VII, an extract of Centella asiatica, madecassic acid, asiatic acid, an oat extract, an extract of Bertholletia excelsa, a hydrolysate of soya proteins or peptides, an extract of Potentilla erecta, an extract of Siegesbeckia orientalis, ginsenosides or notoginsenosides, in particular Rb1, R0, an extract of seeds of Vigna aconitifolia,
molecules or extracts promoting the synthesis of hyaluronic acid or of glycosaminoglycans in the epidermis and dermis, such as an extract Vital Essence of Mamaku, an extract of leaves of Cyathea medullaris, an extract of Eriobotrya japonica or fragments of hyaluronic acid of low molecular weight or an extract of Adenum obesum,
molecules or extracts regulating the differentiation of the epidermis such as ecdysterone, turkesterone, calcium derivatives, vitamin D precursors
adenosine, carnitine or its derivatives in particular acetylcarnitine, vitamin A or one of its cosmetically acceptable esters, in particular the propionate or the palmitate of vitamin A.
inhibitors of metalloproteinases (MMP), in particular inhibitors of MMP 1, 2, 9, such as an extract of Ruscus asculeatus, soya peptides or flavonoids such as quercetin, kaempferol, apigenin, wogonin or vegetable extracts containing them,
inhibitors of elastase such as vegetable extracts of Aspergillus fumigatus, of Momordica charantia, of Cucurbita maxima
substances stimulating the synthesis of dermatopontin, such as an extract of amber,
astringent molecules or extracts of plants that close up the pores such as an extract of Hamamelis
filters protecting against UVA and UVB radiation, such as benzophenone, 4-butyl methoxydibenzoylmethane, ethylhexyl methoxycinnamate, octocrylene, ethylhexyl salicylate, sulphonic acid phenylbenzimidazole, homosalate, alone or in combination with oxides of titanium
molecules or vegetable extracts acting on the pigmentation such as kojic acid, extracts of liquorice or mulberry root, arbutin, calcium pantothenosulphonate, boldine, diacetylboldine, vitamin C or one of its derivatives, such as glycosides, extracts of lily, in particular of the bulb.
antiradical or anti-inflammatory molecules or extracts such as an extract of Artemisia capillaris, an extract of Sanguisorba officinalis, resveratrol and its derivatives, curcuma, curcumin or tetrahydrocurcumin, polyphenols extracted from grapeseed, vitamin E and its derivatives, in particular its phosphate derivatives, ergothioneine or its derivatives, idebenone, piceid, stilbenes or hydroxyphenanthrenes.
an extract of orchid such as an orchid belonging to the genus Brassocattleya, for example an extract of the orchid Brassocattleya marcella, or to the genus Vanda, for example an extract of an orchid selected from Vanda coerulea, Vanda teres or Vanda denisoniana.
hydrating molecules such as glycerol or natural polyols, natural or synthetic ceramides, spring or mineral waters.

Advantageously, the composition further comprises at least one cosmetically acceptable excipient, which can be selected from pigments, dyes, polymers, surfactants, rheology agents, perfumes, electrolytes, pH adjusters, antioxidants, preservatives, and mixtures thereof.

The cosmetic composition is advantageously in the form of a serum, a lotion, a cream or a hydrogel, preferably a mask, or in the form of a stick or a patch.

The compositions of the invention have a particularly desirable effect for preventing or delaying the appearance of the signs of aging of the skin or slowing the effects thereof, in particular for restructuring the epidermis, toning the skin, and/or promoting the attenuation or resorption of wrinkles.

As already mentioned, the present invention relates to a use of clover honey as defined above as a cosmetic active agent intended for preventing or delaying the appearance of the signs of aging of the skin or slowing the effects thereof, in particular intended for restructuring the epidermis, toning the skin, and/or promoting the attenuation or resorption of wrinkles.

The invention also relates to a method of cosmetic care, characterized in that it comprises the application, on the areas of skin in question, of clover honey or a cosmetic composition as defined previously, in an effective amount for preventing or delaying the appearance of the signs of aging of the skin or slowing the effects thereof, in particular for restructuring the epidermis, toning the skin, and/or promoting the attenuation or resorption of wrinkles.

Advantageously, application takes place on an area of skin of the face, of the neck or of the body, notably of the hands, having signs of aging such as the presence of wrinkles or lines.

For each of the objects of the aforementioned invention, the concentration of clover honey used as cosmetic active agent will be between 0.001 and 10 wt. %, more particularly between 0.01 and 5 wt. %, better still between 0.1 and 3 wt. %, relative to the weight of the cosmetic composition.

Other aims, characteristics and advantages of the invention will become clear from the explanatory description that now follows, referring to examples of preparation of extracts and of tests demonstrating the properties of the extracts and to an example of a cosmetic composition using said extracts, given purely for purposes of illustration and which therefore do not in any way limit the scope of the invention.

In the examples, all the percentages are by weight, the temperature is in degrees Celsius, pressure is atmospheric pressure, unless stated otherwise.

### DESCRIPTION OF THE DRAWINGS

FIGS. 1a, 1b, 2a to 2f and 3 relate to example 1 given below. FIGS. 4 to 6 relate to example 2.

FIGS. 1a and 1b are two examples of observations made with a phase contrast microscope, of one and the same artificially “injured” zone of the well in which normal human fibroblasts (NHF) are cultivated. The image in FIG. 1a shows a cell-free zone, corresponding to the place where the IBIDI culture insert was located, immediately after withdrawal of said insert. FIG. 1b shows the same zone after partial recolonization by NHF.

FIGS. 2a to 2d show the four stages of analysis of images recorded in blue fluorescence and in red fluorescence, for characterizing the colonization, by fibroblasts, of the zone of the support damaged artificially by means of the IBIDI inserts. FIGS. 2c and 2d show respectively the untreated damaged zone (negative control) after labelling of the cell nuclei (stage 3 of image analysis), then after automatic detection of the cell nuclei (stage 4 of image analysis). FIGS. 2e and 2f show the damaged zone after treatment with a clover honey according to the invention, respectively after stage 3 (FIG. 2e) and stage 4 (FIG. 2f) of image analysis.

FIG. 3 shows on the ordinate, in the form of a bar chart, the number of cells that have recolonized the scar per square centimetre obtained with each of the products tested, respectively the untreated control, designated NT; the commercial product “Gatuline Skin Repair” as positive reference control; thyme honey as another reference product, and the clover honey according to the invention;

FIG. 4 shows on the ordinate, in the form of a bar chart, the ratio of the transcripts of the HMGB1 gene between the normal NHK (Normal Human Keratinocytes) cells treated and the untreated normal NHK cells, normalized with beta-2-microglobulin, obtained with each of the products tested, respectively the control, designated NT; the commercial product “Gatuline Skin Repair” as positive reference control and the clover honey according to the invention;

FIG. 5 shows on the ordinate, in the form of a bar chart, the ratio of the transcripts of the TGF-beta gene between the treated NHF cells (Normal Human Fibroblasts) and the untreated NHF cells, normalized with beta-2-microglobulin; obtained with each of the products tested, respectively the control, designated NT; the commercial product “Gatuline Skin Repair” as positive reference control and the clover honey according to the invention;

FIG. 6 shows on the ordinate, in the form of a bar chart, the ratio of transcripts for gene expression of beta-1,4-galactosyltransferase, between the treated NHF and the untreated NHF, normalized with beta-2-microglobulin, obtained with each of the products tested, respectively the control, designated NT; the commercial product “Gatuline Skin Repair” as positive reference control and the clover honey according to the invention.

### EXAMPLES OF THE INVENTION

### Example 1

### Investigation of Clover Honey in a Model of Healing In Vitro

This experiment is based on the use of biocompatible cell culture inserts permitting quantitative and perfectly reproducible measurement of the phenomena of healing in vitro.

Material and Methods

1. Cell Culture

The cells used in this study are normal human fibroblasts (NHF).

The cells are seeded in flasks of 175 cm² at a density of 1 million per flask in DMEM (Dulbecco's Modified Eagle's Medium) culture medium supplemented with 10% of fetal calf serum and L-glutamine 1.3 mM. At confluence, the cells are trypsinized and reseeded for the healing test.

2. Healing Test

Seeding the Cells

Before seeding the cells, inserts marketed in the form of kits under the name Culture-Insert (ref. 80209) by the company IBIDI (Germany) are placed at the bottom of six wells of a plate. The NHF are seeded at a rate of 20000 cells/cm² in DMEM medium containing 10% of fetal calf serum and cultivated for 48 h.

When the cells reach confluence, the culture medium is changed and replaced with DMEM only. Culture is continued for 24 h.

Treatments

In this study, two identical experiments were performed at an interval of a week in the same conditions. Two wells are used for each substance tested.

The substances tested are as follows:

| GATULINE SKIN REPAIR (positive control) | 0.10% | |
|---|---|---|
| Thyme honey (origin: France) | 100 | μg/ml |
| Clover honey (origin: New Zealand) | 100 | μg/ml |

A cell culture is also performed in DMEM medium alone, without adding any test substance, in two wells, as an untreated control (NT).

GATULINE SKIN REPAIR is an aqueous-alcoholic extract of wild acanthus, marketed by the company GATTEFOSSE (France), which is described as having a protective effect on damaged skin, by stimulating the expression of markers of keratinocyte differentiation (test ex-vivo on stripped skin explants).

The active agents tested (thyme honey and clover honey) are prepared as follows: a stock solution at 100 mg/ml of honey in PBS is prepared, and is then sterilized on a 0.22 μm filter. Final dilution (1/1000th) is in DMEM alone.

Healing

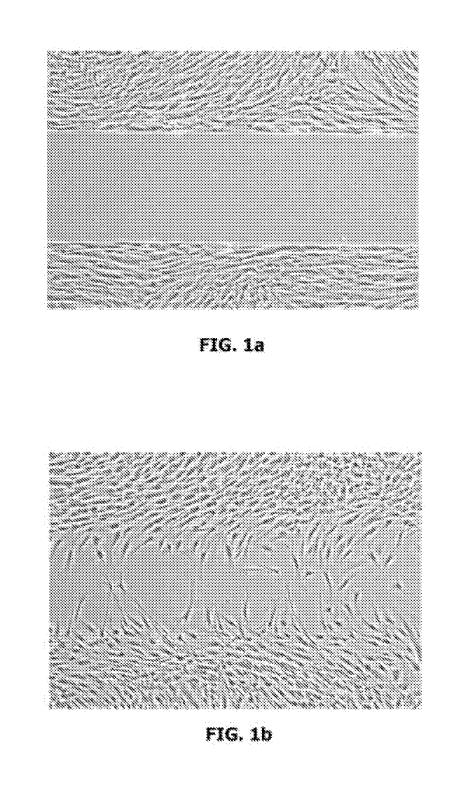

The culture insert is first removed with sterile tweezers. Using a phase contrast microscope, a “damaged” zone is observed, completely devoid of cells, at the place where the insert was located (FIG. 1a).

The initial culture medium is then replaced in each well with the culture medium containing the active agents being tested according to the conditions described above.

The cells are kept in culture for 16 h (optimum measurement time, determined previously), in a stove at 37° C. and in an atmosphere containing 5% CO₂. The rate of recolonization of the cicatricial zone, depending on the treatments, is observed using an image analysis technique (FIG. 1b).

Cell Labelling

After rinsing with PBS, the cells are fixed for 10 minutes with a 10% formalin solution and then rinsed with PBS. The cells are then permeabilized with a 0.1% solution of Triton X100 for 10 minutes.

A solution containing DAPI (4′,6-diamidino-2-phenylindole), a fluorescent molecule used for labelling cell nuclei, and Phalloidine 546, used for labelling the actin cytoskeleton, is deposited on the cells and incubated at room temperature, away from the light for 1 h.

The cells are then rinsed with PBS and covered with a drop of aqueous mounting medium.

Images are immediately obtained on a Nikon TE2000 videomicroscope in blue fluorescence (cell nuclei) and red fluorescence (actin filaments) at a rate of 2 photos per well at the damaged zone.

Image Analysis

Image analysis is used for visualizing and quantifying the colonization by fibroblasts of the artificially “damaged” zone after removal of the insert. Analysis is performed using the Leica QWIN software.

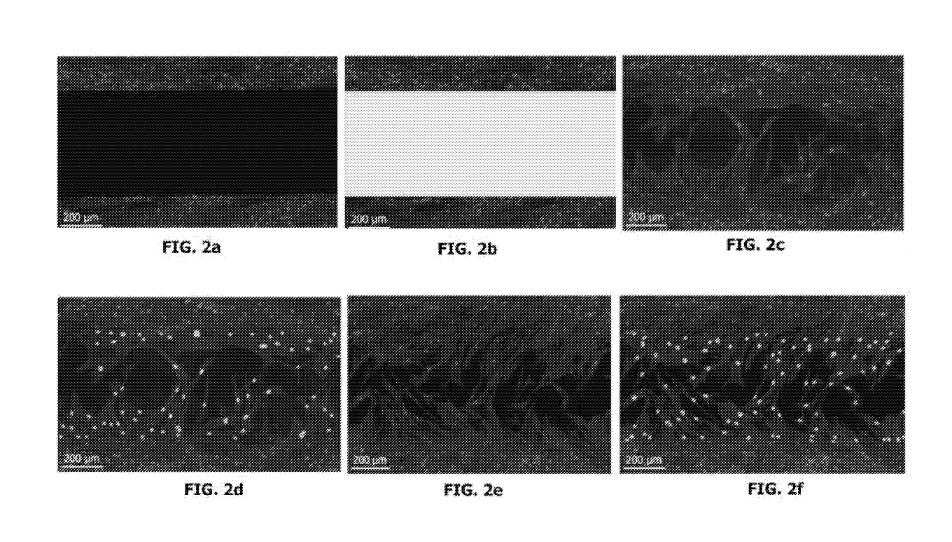

Analysis comprises the following four stages:
1—capture of an image before treatment (time 0), by means of the NIKON TE2000 videomicroscope equipped with NIS software (FIG. 2a): the image shows the stripped zone, devoid of cells, after withdrawal of the insert
2—determination of the measurement zone by computer—zone in yellow (FIG. 2b).
3—capture of a new image after the stages of treatment and labelling of the cell nuclei (FIGS. 2c and 2d for the control NI and FIGS. 2e and 2f for the treatment with the honey of the invention): the damaged zone comprises fibroblasts, which recolonize the initially cell-free zone.
4—automatic selection of the cell nuclei labelled in the zone defined in stage 2, and counting of said labelled cell nuclei (FIGS. 2d and 2f).

The number of cells that have recolonized the damaged zone, per unit area (cm²), is calculated. The measurement is reproduced on 4 images per active agents tested.

3. Results

The effect of each of the active agents tested, on recolonization of the scar, is shown in FIG. 3.

It can clearly be seen from this figure that the clover honey has significant activity on recolonization of the damaged zone by the fibroblasts, of the same order as Gatuline Skin Repair (positive control). In contrast, the clover honey itself produces an effect that is far superior to that of thyme honey, the healing activity of which is well known, which constitutes an unexpected result.

This justifies the choice of clover honey as a cosmetic active agent that is particularly effective for preventing or delaying the appearance of the signs of aging of the skin or slowing the effects thereof.

### Example 2

### Effect of Honey on Expression of Genes Coding for Markers of Healing

The purpose of this study is to evaluate the modulation of the expression of three genes coding for proteins involved in the healing process, by a treatment comprising clover honey.

The genes selected are as follows:
HMGB1 (High Mobility Group Box 1): this gene codes for a multifunctional cytokine implicated in the inflammatory response and healing. A high level of expression of this protein reflects good quality of healing (Straino et al., J. Invest. Dermatol. 2008 June; 128(6):1545-53).
Beta-1,4-galactosyl transferase: this gene codes for a galactosyl transferase that participates in the deposition of collagen, a fundamental component in healing (Shen et al., Am J Dermatopathol. 2008 February; 30(1):10-5).
TGF beta: this gene has a central role in the entire healing process (Wang X J et al., J. Investig. Dermatol. Symp. Proc. 2006 September; 11(1):112-7).

The influence of each active agent on expression of the genes is investigated by quantitative PCR, after a phase of treatment of normal human keratinocytes (NHK) or of normal human dermal fibroblasts (NHF) with the active agent of the invention.

1. Cell Culture

The skin cells (keratinocytes or fibroblasts) are stored in cryotubes immersed in liquid nitrogen (−180° C.), until they are used. After thawing at 37°, the contents of each cryotube are taken up and then centrifuged (5 minutes at 150 g at room temperature).

The cell pellet is taken up in 10 mL of a culture medium appropriate to each type of cells, then seeded in a 75 cm² bottle.

The composition of these media is as follows:

Medium A, Suitable for the Culture of Keratinocytes
KSFM (Invitrogen)+pituitary extracts (Invitrogen) 50 μg/ml

Medium B, Suitable for the Culture of Fibroblasts
DMEM (Invitrogen)+fetal calf serum (Biowest) 10% v/v

At confluence, the cells (keratinocytes or fibroblasts) are detached from the culture support with a trypsin-EDTA medium (1 mL of 0.5% trypsin and 0.2 g/L of EDTA) for a few minutes at 37° C. After neutralizing the action of the trypsin and centrifuging again, each cell pellet is taken up in 1 mL of medium A (NHK) or of medium B (NHF).

2. Treatments

The cells (NHK or NHF) are seeded in a Petri dish of 35 mm diameter at a rate of 2.5×10⁵ cells/dish in their respective medium. The culture medium of the NHF (medium B) is replaced 24 hours before the phase of treatment with the DMEM medium alone.

Two dishes are seeded per culture condition, i.e. 2 dishes for each substance tested and 2 dishes seeded with the culture medium (untreated control), if necessary.

Substances Tested (Dilution in the Culture Medium)

| Clover honey | 100 μg/mL |
|---|---|
| GATULINE SKIN REPAIR (positive control) | 0.01% |

After 24 hours of treatment, the cells are recovered for extracting their total RNAs.

3. Obtaining the Total RNAs by Means of the EZ1 Extractor (Qiagen)

The culture medium of the cells is removed, then 500 μL of Qiazol (supplied in the kit) is added. The cellular lysate is recovered in a 1.5 mL tube. The total RNAs are extracted according to the supplier's protocol.

The solutions of total RNAs obtained are assayed, and their quality is verified, by means of the Bioanalyser 2100 (Agilent Technologies) connected to a computer possessing the special software for analysing the results (software 2100 expert). The technique requires a 12-well microplate (RNA 6000 NanoChips) and a kit of reagents (RNA 6000 Nano Reagents & Supplies), specific for assaying eukaryotic total RNAs.

4. Synthesis of Complementary DNAs

The reverse transcription (RT) kit used is the High capacity cDNA archive kit. It was used according to the protocol supplied. 100 ng of total RNAs, after DNAse, are diluted in water to a final volume of 25 μL. They are then incubated for 10 minutes at 25° C. and then 2 hours at 37° C. with 25 μL of reaction mixture of High capacity cDNA archive kit 2X previously prepared as indicated below.

Reaction mixture High capacity cDNA archive kit 2X for 1 reaction:

| | | |
|---|---|---|
| RT buffer | 5 | μL |
| dNTP buffer | 2 | μL |
| Random primer | 5 | μL |
| RNAse out | 0.5 | μL |
| RT | 2.5 | μL |
| H₂O | 10 | μL |

5. Real-Time Quantitative RT-PCR

The effect of the various treatments is evaluated by real-time quantitative PCR with 96-well fast block of the 7900HT (Applied biosystems).

Preparation of the Reaction Mixture High for 1 Reaction:

| | | |
|---|---|---|
| TaqMan Fast universal PCR master mix (2×) | 10 | μL |
| TaqMan gene expression assay | 1 | μL |
| H₂O | 4 | μL |

The 15 μL of the mixture is placed in the wells of a 96-well plate specially designed for the 7900HT apparatus, 5 μL of water is added (for the blank) or 5 μL of successive dilutions of cDNA (for the range) or 5 μL of samples diluted to 1/50th in the corresponding wells.

Results

Expression of the HMGB1 Gene in NHKs after 24 Hours of Treatment

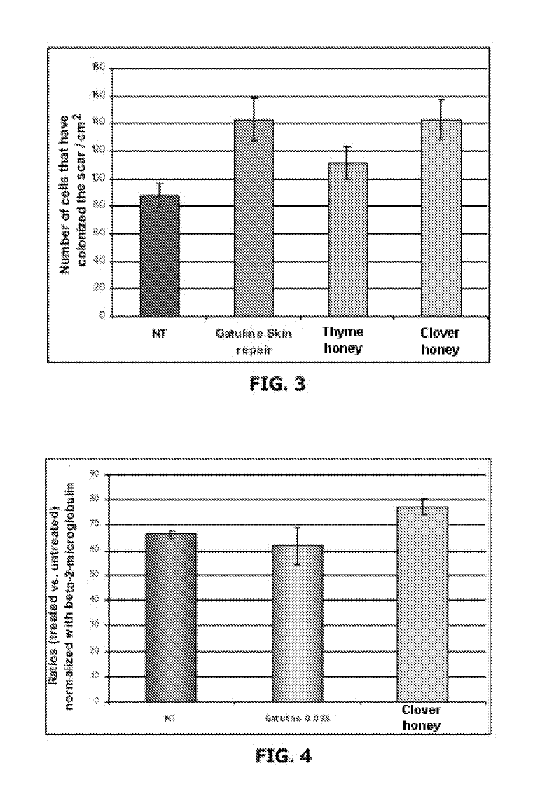

The proportion of transcripts coding for the HMGB1 gene measured for a sample is referred to the proportions of transcripts coding for the invariant gene beta-2-microglobulin (Beta-2-m). The results obtained are processed in the form of histograms (FIG. 4). The treatment with clover honey increases the expression of the gene by a factor of 1.17 relative to the untreated control, in contrast to the positive control, which does not increase the expression of said gene.

Expression of the TGF-Beta Gene in NHFs after 24 Hours of Treatment

The proportion of transcripts coding for the TGF-beta gene is referred to the proportion of transcripts coding for the invariant gene Beta-2-microglobulin (beta-2-m). The results obtained are processed in the form of histograms (FIG. 5). The treatment of cells with clover honey increases the expression of the gene under investigation, by a factor of 1.76 relative to the untreated control. The positive control also significantly increases expression of the gene under investigation.

Expression of the beta-1,4-galactosyltransferase Gene in NHFs after 24 Hours of Treatment

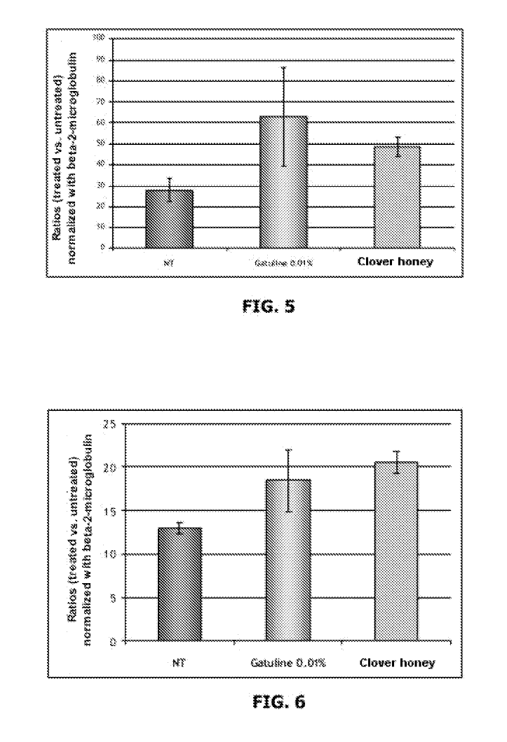

The proportion of transcripts coding for the beta-1,4-galactosyltransferase gene is referred to the proportion of transcripts coding for the invariant gene Beta-2-m. The results obtained are processed in the form of histograms (FIG. 6). After treatment with clover honey, an increase of the proportion of transcripts of the beta-1,4-galactosyltransferase-1 gene relative to the untreated control by a factor of 1.59 is observed. The positive control is also significantly active.

Conclusions

Clover honey significantly stimulates the expression of the genes coding for the markers HMGB1, TGF-beta and beta-1,4-galactosyltransferase.

It promotes all of the processes involved in tissue repair and healing.

This justifies the choice of clover honey as a cosmetic active agent that is particularly effective for preventing or delaying the appearance of the signs of aging of the skin or slowing the effects thereof.

### Example 3

### Anti-Aging Serum Improving the Firmness of the Skin

A serum is prepared with the following composition (percentages by weight relative to the final composition):

| | |
|---|---|
| Clover honey | 4 |
| Royal jelly | 0.5 |
| Heterosides of Centella asiatica | 0.1 |
| Extract of Malva sylvestris | 0.1 |
| Extract of Vigna aconitifolia | 0.1 |
| Excipients, including preservatives | q.s. 100 |

The serum is applied on the face in the morning to improve the firmness of the skin.

### Example 4

### Soothing Anti-Aging Night Cream

An emulsion is prepared with the following composition (percentages by weight relative to the final composition):

| | |
|---|---|
| Glycolysate of clover honey | 1 |
| (for example hydroglycolic | |
| (water-butylene glycol mixture)) | |
| Glycerol | 3 |
| Mango butter | 2 |
| Hyaluronic acid | 1 |
| Ergothioneine | 0.4 |
| Ascorbyl glycoside | 0.4 |
| Alpha-tocopherol | 0.2 |
| Potassium glycyrrhizinate | 0.05 |
| Preservatives | qs |
| Excipients perfumed emulsion | q.s. 100 |

The cream is applied at bedtime, on the face, in particular on areas with signs of skin aging.

1. A method for preventing or delaying the appearance of the signs of aging of the skin or slowing the effects thereof, wherein said method comprises the topical application on the skin of a honey from clover of the species Trifolium repens, or an extract of said honey, as active agent in a cosmetic composition.

2. The method of claim 1, wherein said method is for restructuring the epidermis, for toning the skin, or for promoting attenuation or resorption of wrinkles or skin lines.

3. The method of claim 1, wherein said honey from clover of the species Trifolium repens, or an extract thereof is used as a cosmetic anti-wrinkle agent.

4. The method of claim 1, wherein said cosmetic composition comprises from 0.01 to 10 wt. % of clover honey of the species Trifolium repens.

5. The method of claim 1, wherein said cosmetic composition comprises from 0.01 to 5 wt. % of clover honey of the species Trifolium repens.

6. The method of claim 1, wherein said cosmetic composition comprises one or more unifloral honeys from other plants.

7. The method according to claim 6, wherein said unifloral honey is selected from the group consisting of a unifloral honey from thyme (Thymus vulgaris), a honey from manuka (Leptospermum scoparium), one or more polyfloral honeys, one or more extracts of the honeys, one or more products from apiculture, royal jelly, and any mixture thereof.

7. A Cosmetic composition comprising, as cosmetic active agent, a honey from clover of the species Trifolium repens or an extract thereof.

8. The composition according to claim 7, wherein said composition is an anti-aging or anti-wrinkle composition comprising said clover honey or an extract thereof, as anti-wrinkle or anti-aging active agent.

9. The composition according to claim 7, wherein said composition comprises from 0.01 to 10 wt. % of clover honey of the species Trifolium repens or an extract thereof.

10. The composition according to claim 6, wherein said composition comprises from 0.01 to 5 wt. % of clover honey of the species Trifolium repens or an extract thereof.

11. The composition according to claim 7, wherein said composition comprises one or more unifloral honeys from other plants.

12. The composition according to claim 11, wherein said unifloral honey is selected from the group consisting of a unifloral honey from thyme (Thymus vulgaris), a honey from manuka (Leptospermum scoparium), one or more polyfloral honeys, one or more extracts of the honeys, one or more products from apiculture, royal jelly, and any mixture thereof.

13. The composition according to claim 7, wherein said composition is a serum, a lotion, a cream, a hydrogel, a mask, a stick, or a patch.

14. A method of cosmetic care, wherein the method comprises application, on areas of skin in question, of a clover honey of the species Trifolium repens or an extract thereof, or of a cosmetic composition as defined in claim 7, in an effective amount for providing at least one of the following cosmetic care selected from the group consisting of preventing or delaying the appearance of the signs of aging of the skin, slowing the effects thereof, restructuring the epidermis, toning the skin, and promoting attenuation or resorption of wrinkles.

15. The method according to claim 14, wherein the method comprises an application of said clover honey of the species Trifolium repens or extract thereof, or of said cosmetic composition on an area of skin of the face, of the neck, of the hands having signs of aging, or on a area presenting wrinkles or skin lines.
